(19) United States
(12) Patent Application Publication
(10) Pub. No.: US 2014/0107576 A1
(43) Pub. Date: Apr. 17, 2014

(54) APPARATUS FOR LEFT-SIDE INSTALLATION OF TUNNELED VASCULAR CATHETERS

(71) Applicant: University of Manitoba, Winnipeg (CA)

(72) Inventor: Sean ARMSTRONG, Winnipeg (CA)

(73) Assignee: University of Manitoba, Winnipeg (CA)

(21) Appl. No.: 14/056,406

(22) Filed: Oct. 17, 2013

(30) Foreign Application Priority Data
Oct. 17, 2012 (CA) 2792682

(51) Int. Cl. A61M 25/09

(52) U.S. Cl. CPC A61M 25/09041
USPC 604/104

(57) ABSTRACT

An apparatus for installation of a tunneled vascular catheter into a patient's right atrium from the left side of their upper body. The apparatus comprises a dilator and a peel-away sheath. The dilator has a distal end portion and a proximal end portion interconnected by a shaft portion, with a lumen extending therethrough. The distal end portion is curved about 1° to about 3° from the longitudinal axis of the shaft portion. The end of the proximal end portion is provided with a flange. The total length of the dilator is about 3 cm to about 6 cm. The distal end of the peel-away sheath is provided a flange for contacting the flange at the end of the proximal end portion of the dilator. The length of the peel-away sheath is about 2.5 cm to about 4.5 cm.

### TECHNICAL FIELD

Various embodiments disclosed herein generally relate to apparatus for installation of tunneled vascular catheters to provide regularly scheduled hemodialysis treatments. More specifically, this disclosure pertains to apparatus for installation of tunneled vascular catheters into patient's right atrium using their left brachiocephalic vein as the delivery route.

### BACKGROUND

Hemodialysis is a regularly scheduled clinical procedure for patients suffering from kidney failure. For each procedure, blood is removed from the patient and transported to a dialysis machine where toxins are removed by diffusion through a semi-permeable membrane into a dialysis fluid. The filtered blood is then returned through the catheter body to the patient. Depending on the severity of the patient's condition, the hemodialysis treatments can be administered several times per week or as often as a daily basis. Because of the closely spaced treatment intervals, dialysis patients require some form of easily accessible to their vascular system. The most common routes are fistulas, grafts, and tunneled vascular catheters.

Estimates suggest that up to 50% of patients requiring hemodialysis treatments have had tunneled vascular catheters installed. The National Kidney Foundation Kidney Disease Outcomes Quality Initiative recommends that the preferred site for installing a tunneled vascular catheter is into the right internal jugular vein because of the ease of ingress and installation with minimal patient perturbation and mis-adventure. However, prolonged repeated administration of hemodialysis treatments often results in the occurrence of thrombosis in the right jugular vein and/or infections occurring around the catheter installation site. Estimates suggest that up to 40% of tunneled vascular catheter failures are due to venous thrombosis or the formation of fibrin sheaths about the catheters. Furthermore, it is estimated that infection occurs in over 50% of tunneled vascular catheter installations, and that patient complications arising from about two thirds of the infected catheters results in loss of the right jugular vein for routine hemodialysis procedures. In such cases, it is necessary to access the right atrium of the patient's heart through the left side of a patient's body i.e., via the left brachiocephalic vein. The problem with left-sided catheter placement is the increased risk of serious life-threatening vascular accidents because, compared to the direct path into the right atrium provided by the right jugular vein, the left brachiocephalic vein follows a tortuous path around the superior aspect of the heart into the superior vena cava and then into the right atrium. Compounding this problem is that the apparatus used for installing vascular catheters into the right jugular vein, i.e., a dilator encased by a tear-away sheath, is too long and stiff for precisely and delicately accessing and penetrating the left brachiocephalic vein from the patient's left side. Furthermore, the length of the apparatus projecting from the incision site interferes with a surgeon's ease of manipulation of the dilator, the tear-away sheath and the guidewire into the left brachiocephalic vein, and then their ease of manipulation of the catheter through the left brachiocephalic vein into the patient's right atrium.

### SUMMARY OF THE DISCLOSURE

The exemplary embodiments of the present disclosure pertain to apparatus for installation of tunneled vascular catheters into a patient's right atrium from the upper left side of their body. The apparatus generally comprises a dilator with a lumen therethrough along the longitudinal axis and a peel-away sheath for encasing the shaft and proximal end of the dilator while leaving the distal end of the dilator exposed. The dilator has a total length of about 3 cm to about 5 cm. The length of the peel-away sheath is about 2.5 cm to about 4.5 cm.

The apparatus may additionally comprise a guidewire for traversing the lumen, wherein the guidewire has a first stiffness when a pushing force is applied to the guidewire, and a second stiffness when a pulling force is applied to the guidewire. The second stiffness provides more flexibility to the guidewire than is provided by the first stiffness, to facilitate removal of the guidewire from the patient's body after the vascular catheter has been installed.

### BRIEF DESCRIPTION OF THE FIGURES

The drawings described herein are for illustrative purposes only of selected embodiments and are not intended to limit the scope of the present disclosure.

FIG. 1(A) shows an illustration of an exemplary assembled prior art peel-away sheath/dilator apparatus, and 1(B) shows an illustration of an exemplary peel-away sheath/dilator apparatus according to an embodiment of the present disclosure;

FIG. 2(A) is an illustration of an installation of a prior art peel-away sheath/dilator assembly into a patient's right jugular vein, and 2(B) is an illustration of a tunneled vascular catheter installed with the assembly shown in 2(A);

FIG. 3 shows an illustration of the components comprising an exemplary apparatus according to an embodiment of the present disclosure;

FIG. 4 is an illustration of an installation of an exemplary dilator/peel-away sheath assembly into a patient's left brachiocephalic vein using the left jugular vein for access; and

FIG. 5 is an illustration of a tunneled vascular catheter installed with the assembly shown in FIG. 4.

### DETAILED DESCRIPTION

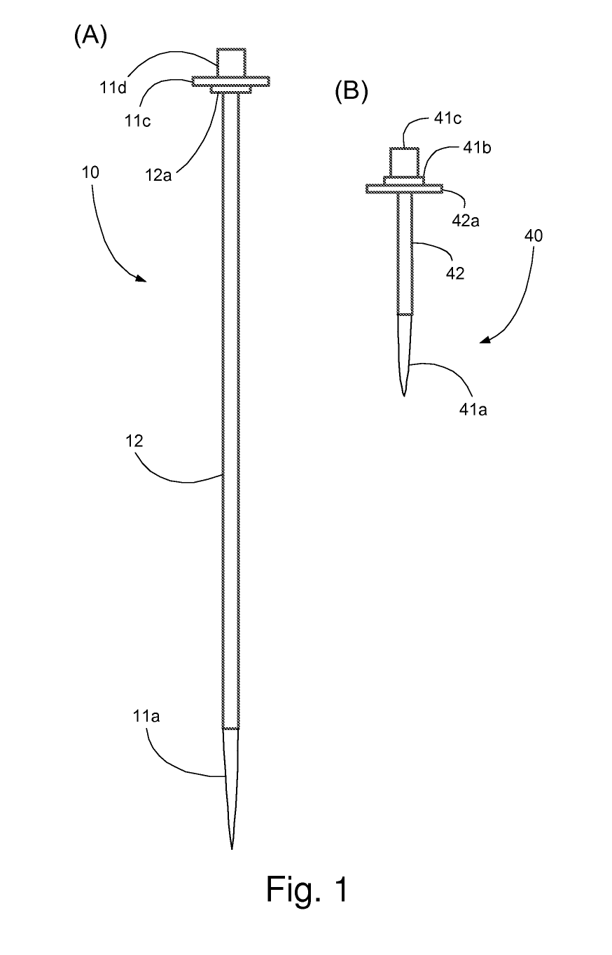

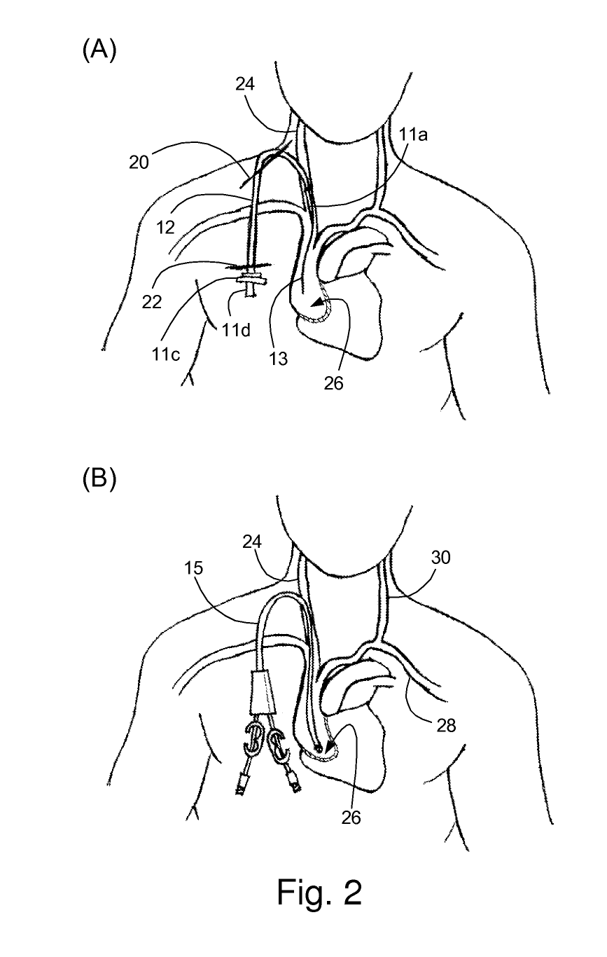

Apparatus and procedures for installing tunneled vascular catheters into a patient's right jugular vein are well-known and are generally based on Seldinger's technique. Briefly, Seldinger's technique comprises making a downward incision into the lower right side of the patient's neck area just above about the upper edge of their right clavicle for insertion of a sharp hollow needle called a trocar into the right jugular vein, typically using a visualization aid such as ultrasound imagery, luminescence, fluoroscopy, and the like. A round-tipped guidewire is then advanced through the lumen of the trocar into the jugular vein, after which the trocar is removed. A second incision is made upward into the patient's upper right breast area several centimeters below the first incision. The distal end of the guidewire extending out from the first incision, is inserted downward underneath the patient's skin layers and is tunneled to the second incision from which it is recovered. The distal end of the guidewire is inserted into and through a tunneling dilator assembly comprising a dilator with its shaft encased within a peel-away sheath, via a lumen extending through the length of the dilator. Dilators commonly used for this type of procedure range in length from about 20 cm to about 25 cm, and generally comprise a relatively stiff material that has some flexibility to enable bending into a U-shape to negotiate the turn at the first incision site toward the right jugular vein. The commonly used dilators are typically made of polymeric materials exemplified by polyethylene, polyurethane, polystyrene, and the like. The tip of the dilator is exposed and facilitates tunneling of the encased dilator upward over the guidewire from the second incision site to the first incision site, then bent into a U-shape and further tunneled from the first incision site into the right jugular vein, and then downward within the jugular vein toward the patient's right atrium. An exemplary prior art peel-away sheath-encased dilator assembly 10 is shown in FIG. 1(A) and typically ranges in length from about 17 cm to about 20 cm, so that the distal tip of the dilator is exposed the peel-away sheath is installed over the dilator. The tapered dilator tip 11a typically dissects tissue as it is tunneled under the patient's skin from the second incision site 22 to the first incision site 20 to the right jugular vein 24 (FIG. 2(A)). The proximal end 11b of dilator is provided with a flange 11c and a threaded housing 11d to threadingly engage a syringe for flushing the dilator with fluid through the lumen prior to use. The dilator is encased by the peel-away sheath 12 which has a flange 12a at its proximal end. After about 3 cm to about 5 cm of the peel-away sheath 12 is installed into the jugular vein 24, the dilator 11 is carefully removed from the second incision leaving the peel-away sheath 12 installed in place. A vascular catheter 15 is then inserted over the guidewire 13 into the peel-away sheath 12 and is advanced through the peel-away sheath 12 into the right jugular vein 24. The vascular catheter is 15 then delivered into and positioned in the patient's right atrium 26 with the assistance provided by manipulation of the guidewire 13 and imaging exemplified by fluoroscopy and luminescence. Then, the guidewire 13 is carefully removed from the second incision site followed by removal of the peel-away sheath 12, leaving the installed vascular catheter 15 in place. As can be clearly seen in FIG. 2, use of the right jugular vein 24 is the preferred route for installation and delivery of a tunneled vascular catheter into a patient's right atrium due to: (i) the ease of tunneled access to the right jugular vein from the patient's upper right chest area requiring only a simple U-turn near the patient's clavicle, and (ii) a direct downward vertical route from the catheter from the entry point into the right jugular vein to the right atrium.

When hemodialysis access via right jugular vein is lost due to infection and/or thrombosis, it is necessary to install a tunneled vascular catheter into the patient's right atrium from their left side via the left brachiocephalic vein 28 which drains the left jugular vein 30. Accordingly, the procedures for installing a tunneled vascular catheter from a patient's left side requires an incision to be made into the lower left side of a patient's neck area just above about the upper edge of their left clavicle to enable insertion of a trocar into their left jugular vein. The dilator tip of an assembled sheath-encased dilator assembly is inserted into the incision and tunneled into the left jugular vein, then very carefully and cautiously manipulated from the left jugular vein into and through the left brachiocephalic vein into the entrance to the right atrium. A guidewire is then inserted through the lumen of the dilator until the guidewire extends into the right atrium. The dilator is then carefully removed, leaving the sheath in place with its distal end extending into and through the left brachiocephalic vein. The proximal end of the sheath extends out of the incision about 5 cm to about 10 cm. A vascular catheter 15 is then inserted over the guidewire through the incision site into and through the left jugular vein into and through the left brachiocephalic vein into the right atrium where it is positioned into place. The problem is that the left brachiocephalic vein follows a tortuous “M-shaped” route above and around the aorta into the inferior vena cava which drains into the right atrium. The known prior art right-sided dilator/peel-away sheath assemblies are too long and stiff for use for insertion into and manipulation through the patient's left brachiocephalic vein. The stiffness of the dilator/peel-away assemblies makes it almost impossible to navigate the M-shaped route without damaging the left brachiocephalic vein and causing catastrophic trauma to the patient. Furthermore, the tortuous pathway into and through the M-shaped left brachiocephalic vein often results in multiple kinks being formed in the peel-away sheaths after the dilators are removed, as a consequences of the tight turns characterizing (i) the juncture of the left jugular vein and the left brachiocephalic vein, and (ii) the M-shaped pathway of the left brachiocephalic vein. The presence of kinks in the peel-away sheaths further exacerbate the difficulty of installing a vascular catheter without causing trauma and harm to the patients.

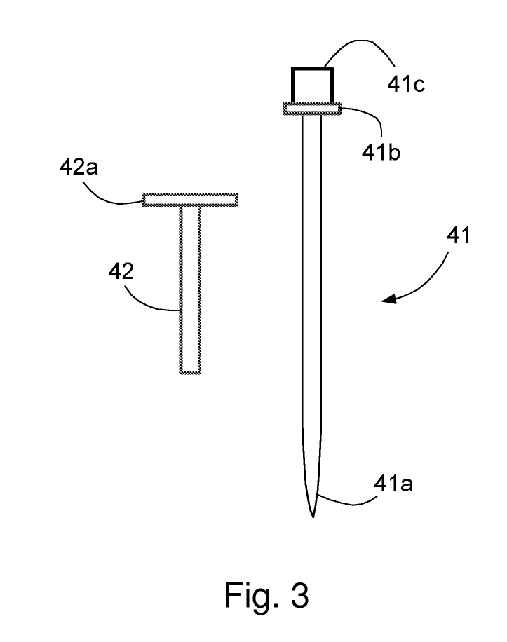

Exemplary embodiments of the present disclosure pertain to apparatus for installation of tunneled vascular catheters from a patient's left side into their left brachiocephalic vein for delivery into the right atrium. An exemplary apparatus 40 is shown in FIG. 1(B) and FIG. 3, and generally comprises: (i) a comparatively very short dilator 41, and (ii) a comparatively very short peel-away sheath 42, in comparison to the common prior art dilator/peel-away sheath apparatus exemplified in FIG. 1(A). The dilator 41 has a distal end with a tunneling tip 41a and a proximal end 41b having an external thread to threadingly receive a syringe for flushing the dilator with fluid through the lumen prior to use, and (ii) a shorter peel-away sheath 42 for encasing the dilator 41. The dilator is about 4 cm to about 6 cm long while the peel-away sheath is about 2.5 cm to about 5 cm long. The diameter of the dilater may be about 4 mm to about 6 mm. The dilator comprises a resiliently flexible material for deflecting and following the contours of the M-shaped left brachiocephalic vein. Because of the relatively short distance from an incision site into a patient's lower neck area to their left jugular vein, the short dilators according to the present disclosure can be more flexible than the relatively long prior art dilators, i.e., over 17 cm long, and therefore, can be made of polymeric materials exemplified by polypropylene, polyethylene, polyvinyl chloride, and blends thereof. Particularly suitable are polymers comprising polytetrafluoroethylene.

Another embodiment pertains to guidewires for use with the exemplary apparatus of the present disclosure. An exemplary guidewire is provided with two degrees of stiffness with the first degree stiffer than the second degree. The guidewires may be from about 25 cm to about 45 cm long. The guidewires exhibit the first degree of stiffness when a pushing force is applied, for example, when pushing a guidewires through the lumen of the dilator. The first degree of stiffness allows more stability for manipulating the guidewire through the left jugular vein into and through the left brachiocephalic vein and into the right atrium. The first degree of stiffness is maintained during installation of the catheter and provides support for catheter while it is manipulated through the left jugular vein into and through the left brachiocephalic vein and into the right atrium. The guidewires exhibit a second degree of stiffness when a pulling force is applied. The second degree of stiffness is more flexible and flaccid in comparison to the first degree of stiffness, and facilitates removal of the guidewire through a lumen of the catheter and out of the patient's body without significant displacement or movement of the cathether during the guidewire removal process. Those skilled in these arts will understand that term “stiffness” is a relative term and can be defined by “Young's modulus” which is a calculation of the ratio of stress to strain. The value of Young's modulus for the first degree of stiffness can be defined “X”. Accordingly, the second degree of stiffness will have a Young's modulus value of about 0.6X, about 0.5X, about 0.4X, about 0.3X, about 0.2X, about 0.1X, about 0.05X, and therebetween.

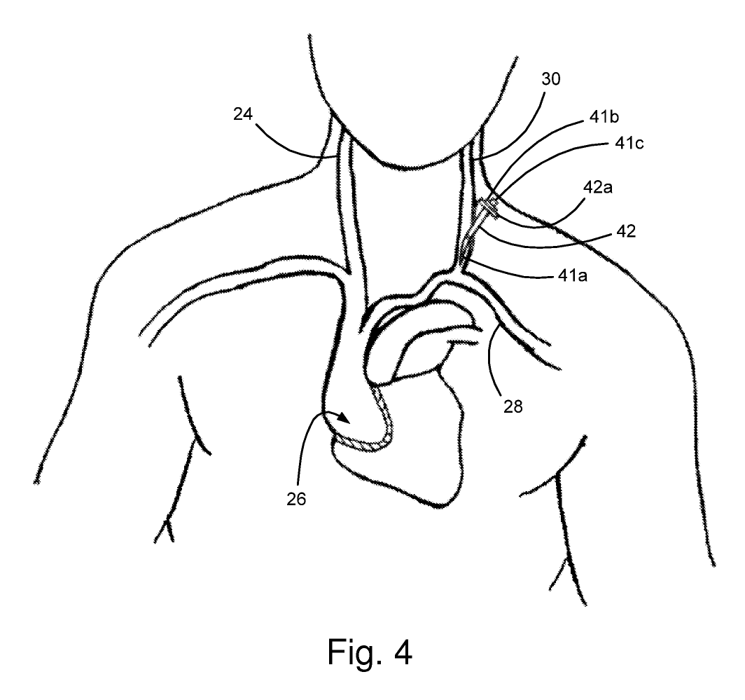

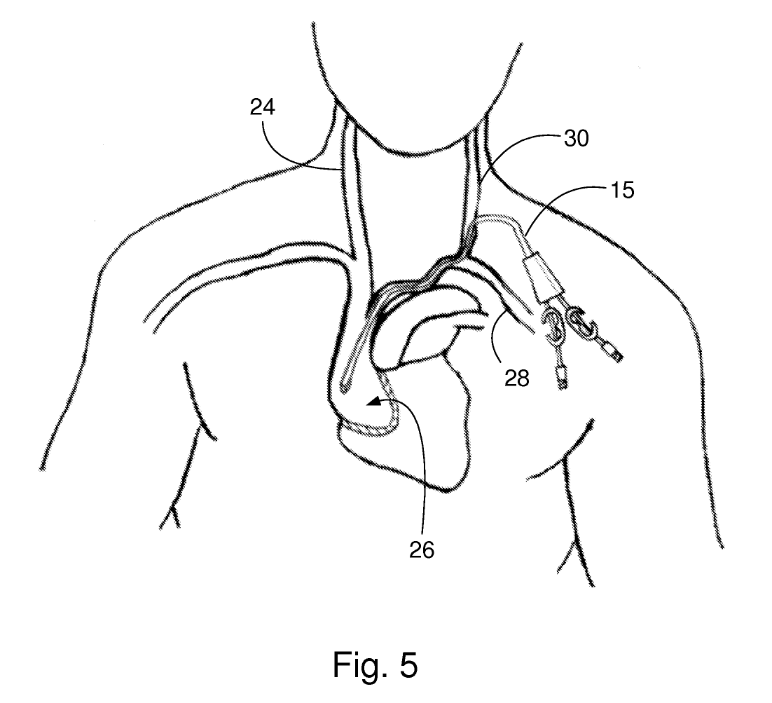

An exemplary method for use of the apparatus of the present disclosure for introduction of a tunneled vascular catheter from a patient's left side and its delivery into their right atrium via the left brachiocephalic vein begins with a downward incision about the upper edge of a patient's left clavicle for insertion of a trocar into their left jugular vein using a visualization aid such as ultrasound imagery, luminescence, fluoroscopy, and the like. The trocar is then removed, and an exemplary apparatus of the present disclosure is inserted into the incision site and then into the patient's left jugular vein 30 (FIG. 4). The length of the dilator 41 (FIG. 3) chosen for insertion into a patient's left jugular vein is selected so that when the flange 41b at the proximal end of the dilator 41 contacts the flange 42a at the proximal end of the peel-away sheath 42 after insertion of the apparatus 40 through the incision into the patient's left jugular vein 30, the distal tip 41a of the dilator 41 terminates in the left jugular vein 30 just prior to the juncture of the left jugular vein with the left brachiocephalic vein 28 (FIG. 4). The length of the peel-away sheath 42 is selected such that its proximal end is positioned in the patient's left jugular vein 30 about 1 cm from the left jugular vein with the left brachiocephalic vein 28. The dilator 42 is then removed from the patient. A vascular catheter 45 is then inserted into the peel-away sheath 42 and manoeuvred using fluoroscopy, through and then out of the sheath 42 into the left jugular vein 30, and then into brachiocephalic vein 28, and then forward into the right atrium 26 (FIG. 5). After the vascular catheter 45 has been installed into the right atrium 26, the peel-away sheath 42 is removed from the patient's body. The catheter 45 is then anchored and the incision site is sutured. It is optional, after the dilator 41has been removed, to also remove the guidewire 13 before introducing the vascular catheter into the peel-away sheath. The vascular catheter 45 can then be manipulated into the left brachiocephalic vein 28 and then into the right atrium 26 using fluoroscopy for visual guidance.

Some right-side catheter installations may prove to be very challenging using the above procedure because of the twists and turns encountered during maneuvering the cathetor through the peel-away sheath 42 into and through the left brachiocephalic vein, and/or into and throught the inferior vena cava, and/or into and positioning in the right atrium. In such cases, the catheter should be removed from the patient's body, and a guidewire inserted into and through the peel-away sheath, and carefully manoeuvred into the patient's right atrium. The proximal end of the guidewire should be inserted into a lumen of a fresh catheter, after which, the catheter is then inserted into the peel-away sheath and carefully manipulated into position into the patient's right atrium. The guidewire is then removed from the patient's body followed by removal of the peel-away sheath.

1. An apparatus for installation of a tunneled vascular catheter into a patient's right atrium from the left side of their upper body, comprising:
a dilator having a distal end portion, a proximal end portion, and a shaft portion connecting the distal end portion and the proximal end portion, a lumen extending therethrough from the distal end portion to the proximal end portion, wherein the end of the proximal end portion is provided with a flange, said dilator having a length from the tip of the distal end portion to the flange of the proximal end portion of about 3 cm to about 6 cm; and
a peel-away sheath for encasing a portion of the dilator, said peel-away sheath having a length of about 2.5 cm to about 4.5 cm, wherein a distal end of the peel-away sheath is provided with a flange for contacting the flange at the end of the proximal end portion of the dilator.

2. The apparatus of claim 1, additionally comprising a guidewire for traversing the lumen, said guidewire having a first stiffness when a pushing force is applied and a second stiffness when a pulling force is applied, wherein the second stiffness provides more flexibility to the guidewire than is provided by the first stiffness.

3. The apparatus of claim 1, wherein the proximal tip portion is resiliently flexible.

4. Use of the apparatus of claim 1 for installation of a vascular catheter into a patient's right atrium, comprising the steps of:
making an incision into the patient's lower neck area about their left clavicle;
inserting the proximal end portion of the dilator into the incision, said dilator encased in the peel-away sheath with the proximal end portion extending from the peel-away sheath;
tunneling the dilator to the patient's left jugular vein and inserting the dilator into the left jugular vein to about the juncture of the left jugular vein with the left brachiocephalic vein until the distal portion of the peel-away sheath contacts the patient's lower neck area about the incision site;
removing the dilator from the patient's body while leaving the peel-away sheath secured in place with its proximal end extending into the left jugular vein toward the left brachiocephalic vein;
inserting a vascular catheter into the distal end of the peel-away sheath and manipulating the catheter into the left brachiocephalic vein and further into the patient's right atrium; and
removing the peel-away sheath from the patient's body.

5. Use of the apparatus of claim 1 for installation of a vascular catheter into a patient's right atrium, comprising the steps of:
making an incision into the patient's lower neck area about their left clavicle;
inserting the proximal end portion of the dilator into the incision, said dilator encased in the peel-away sheath with the proximal end portion extending from the peel-away sheath;
tunneling the dilator to the patient's left jugular vein and inserting the dilator into the left jugular vein to about the juncture of the left jugular vein with the left brachiocephalic vein until the distal portion of the peel-away sheath contacts the patient's lower neck area about the incision site;
inserting a guideline into the lumen at the distal end of the dilator and manipulating the guidewire through the dilator into the left brachiocephalic vein and further into the patient's right atrium;
removing the dilator from the patient's body while leaving the peel-away sheath secured in place with its proximal end extending into the left brachiocephalic vein;
inserting a vascular catheter over the guidewire into the distal end of the peel-away sheath and manipulating the catheter into the left brachiocephalic vein and further into the patient's right atrium;
removing the guidewire from the patient's body by withdrawing said guidewire through the catheter; and
removing the peel-away sheath from the patient's body.
